(19) United States
(12) Patent Application Publication
(10) Pub. No.: US 2025/0360329 A1
(43) Pub. Date: Nov. 27, 2025

(54) DIATHERMY PATIENT WARMING

(71) Applicant: Kenneth L. WILLEFORD, Little River, SC (US)

(72) Inventor: Kenneth L. WILLEFORD, Little River, SC (US)

(21) Appl. No.: 18/669,744

(22) Filed: May 21, 2024

(51) Int. Cl. A61N 1/40; A61B 18/18

(52) U.S. Cl. CPC A61N 1/403; A61B 2018/1876

(57) ABSTRACT

A method of warming a patient during a surgical procedure may use short-wave diathermy applied with an array of adjacent electrical wire loops that create oscillating electromagnetic fields. Oscillating electric and magnetic fields produce heat in biological tissues by inducing a rapidly alternating movement of charged particles within tissues. The method may be implemented using a short-wave signal generator electrically coupled to the array.

### BACKGROUND

### Field of the Invention

The present invention relates to the warming of a patient, e.g., for prevention or treatment of patient hypothermia during a surgical procedure.

### Description of Related Art

Intraoperative hypothermia of surgical patients is a common problem with well-documented adverse events. Unintentional intraoperative heat loss occurs as a result of low ambient temperatures in the operating room, open exposed wounds, administration of cool intravenous fluids, cool irrigating fluids, sterile preparation of the surgical site with fluids creating evaporative losses, and the effect of anesthetic agents that impair the body's ability to thermoregulate by the hypothalamus with concurrent vasodilation increasing heat loss and reduced metabolism with decreased heat production. That is, typically, patients have their skin exposed, are washed with cold soap in a cold room, and are given medications that prevent the normal thermoregulatory mechanisms.

Physiologic effects of hypothermia include decreased oxygen tension in the blood, decreased metabolism, decreased drug biotransformation, impaired renal transport processes, altered membrane excitability, changes in cardiac rate and rhythm, central nervous system depression, hyperglycemia, and sympathetic nervous system stimulation.

Outcome studies have confirmed an increase in wound infection, myocardial infarction, need for post-operative mechanical ventilation, probability for blood transfusion, and mortality in hypothermic versus normothermic patients.

Several methods are currently employed to prevent hypothermia in surgical patients. These include insulation of the patient, humidification of inspired respiratory gases, warming of intravenous fluids, and forced air warming blankets. However, it may be desirable to have a method and system of preventing hypothermia that is more effective and versatile than these existing methods.

### BRIEF SUMMARY OF ASPECTS OF THE DISCLOSURE

Aspects of the present disclosure may provide a unique method of and apparatus for warming a patient to prevent hypothermia in the patient during a surgical procedure under operating room conditions. Such method and apparatus may provide patient warming mediated by the physics principles of short-wave diathermy applied to patients in a large region that may utilize a circuit including an array of adjacent electrical wire loops oriented such that each adjacent section has an opposite direction of the oscillating electromagnetic field.

### BRIEF DESCRIPTION OF THE DRAWINGS

Aspects of the present disclosure will now be described in conjunction with the accompanying drawings, in which:

FIGS. 1A and 1B show illustrative examples of an inductive coil configuration according to aspects of the present disclosure, using the Intellect SWD 100 Model 1600 short-wave diathermy device;

FIG. 2 shows an example of one of the clinical protocols available in the Intellect SWD 100 Model 1600 short-wave diathermy device;

FIG. 3 shows an example of a diathermy device according to aspects of the present disclosure; and

FIG. 4 shows an example of a wiring diagram of an array of adjacent electrical wire loops according to aspects of the present disclosure.

### DETAILED DESCRIPTION OF ASPECTS OF THE DISCLOSURE

Short-wave diathermy is regulated in the Code of Federal Regulations in Title 21, Chapter 1, subchapter H, part 990, subpart F, which discusses Physical Medicine Therapeutic Devices. Short-wave diathermy may be used to apply to specific areas of the body electromagnetic energy in the radio-frequency (RF) bands of 13.56 or 27.12 MHz and is intended to generate deep heat within body tissues for the treatment of selected medical conditions such as relief of pain, muscle spasms, and joint contractures.

Presently, generally accepted uses of short-wave diathermy may include: pain relief; reduction of muscle spasms; decreasing joint stiffness; treatment of contractures; increasing blood flow; treatment of chronic inflammatory conditions; treatment of bursitis; treatment of tenosynovitis; treatment of synovitis; and treatment of chronic inflammatory pelvic disease.

The mechanism of short-wave diathermy is described as oscillating electric and magnetic fields that produce heat in biological tissues by inducing a rapidly alternating movement of ions, rotation of dipolar molecules, and the distortion of non-polar molecules. A movement of ions represents a real flow of current and occurs readily in tissues rich in electrolytes such as blood vessels and muscle. Resistance to this flow may generally lead to heat production.

Short-wave diathermy effects may be divided into thermal and athermal.

Thermal effects may induce vasodilation, elevation of pain threshold, reduction in muscle spasm, acceleration of cellular metabolism, and increased soft tissue extensibility. The athermal effects may be a result of energy absorption by cells from oscillating electrical fields inducing or enhancing cellular activity. They may include increased blood flow, decreased joint pain and stiffness, reduced inflammation, faster resolution of edema, and accelerated wound healing. Short-wave therapy may be delivered either in a continuous mode or a pulse mode. Average outputs of less than 38 W are considered to be nonthermal, whereas higher outputs are thermal. Short-wave diathermy may heat tissue at depths of 3 to 5 cm, and tissue temperature may be controlled by the length of application, with, for example, maximum increases of 4° C. to 6° C.

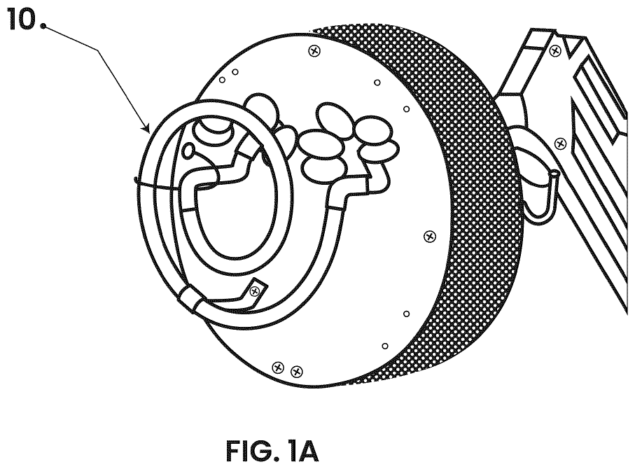

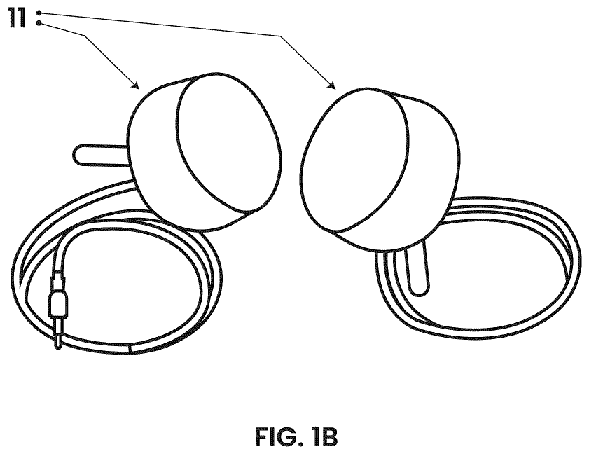

Short-wave diathermy may be applied through the condenser method or by induction. In the condenser method, the treatment site may be placed between two electrodes functioning as capacitor plates; this would be impractical for use as a patient warming system in an operating room because it would limit access to surgical sites, although it may be used in other clinical scenarios. The induction method may utilize a wire 10 that is coiled into a drum 11, an example of which may be seen in FIGS. 1A and 1B. However, even the example shown in FIGS. 1A and 1B may not be sufficient for patient warming because of its relatively small size and thus inability to apply radiation to a significant area of the patient. That is, for purposes of patient warming, which may be used to prevent hypothermia, the above-mentioned depth and intensity of the heating effect may not be effective; rather, it may be better to spread the energy output over a larger area of the patient's body than for the above-mentioned purposes and, for the a given amount of power, achieve a lesser depth of heating and a lower maximum temperature increase, in order to achieve effective patient warming, to help prevent hypothermia (as opposed to treating localized injuries or pain, the uses described above).

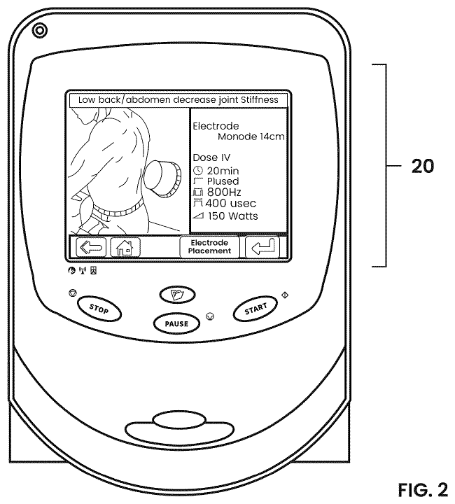

One example of a diathermy device is the Intellect SWD 100 Model 1600 short-wave diathermy device (“the Intellect”), which operates at 27.12 MHz and has 90 clinical protocols, one example of which 20 is shown in FIG. 2. The pulses are typically 20 to 400 usec in duration (pulse width) and are repeated with a frequency of 10 to 800 Hz (pulse frequency). Because the output is pulsed, the average output power levels may be very low (less than 1 W) and still produce an effective treatment. The Intellect SWD 100 in pulsed mode may provide a peak power of 200 W and average powers from a few milliwatts to 64 W.

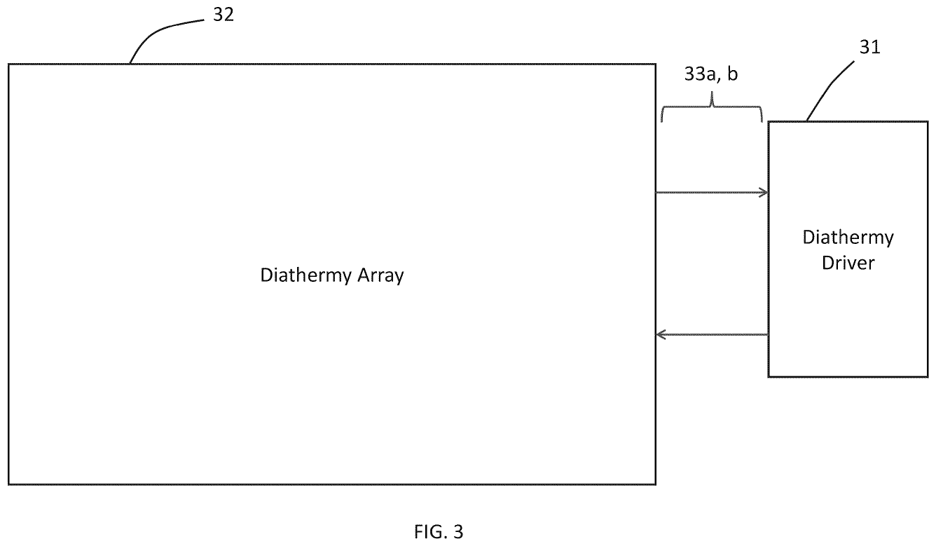

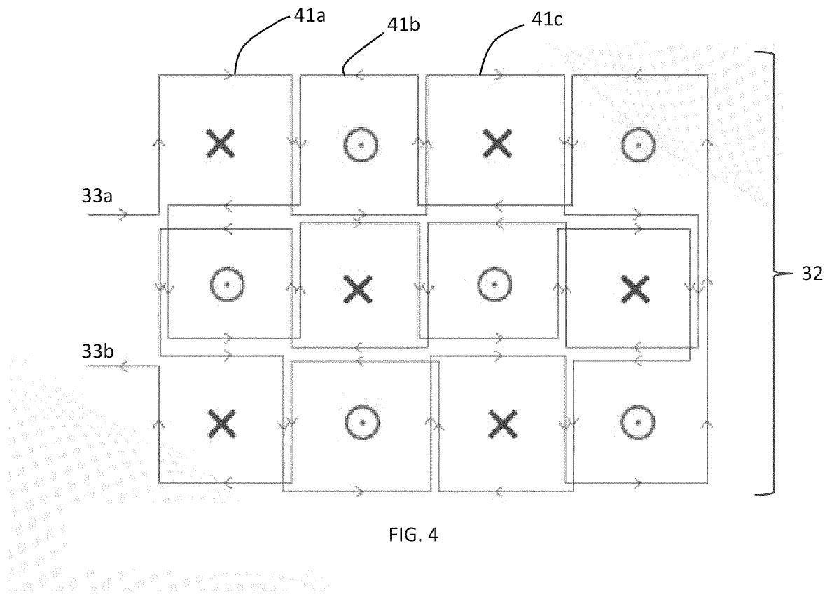

A prototype of an example of a short-wave diathermy patient warming system according to aspects of the present disclosure was built and successfully tested using the Intellect as the signal generator with settings of 800 Hz, 400 microseconds, peak power of 150 watts, and average power of 48 W; FIG. 3 shows a block diagram of the prototype, which shows diathermy driver 31 (which may be the Intellect, as a non-limiting example), a diathermy array 32 (an example of which is described below in conjunction with FIG. 4), and leads 33a, 33b between diathermy driver 31 and diathermy array 32. The impedance of the array 32 and of the Intellect's induction coil were measured at 0.2 Ω.

According to an aspect of the present disclosure illustrated in FIG. 4, and used in the prototype, an array of adjacent electrical wire loops, e.g., 41a, 41b, 41c, may be oriented such that each section has an opposite direction of its oscillating electromagnetic field relative to an adjacent section of a bordering electrical wire loop. The direction of the magnetic field in each section may be determined by the right hand rule. In FIG. 4, the loops containing an “X,” such as loops 41a and 41c, may generate magnetic fields directed into the plane of the page (i.e., containing FIG. 4), while the loops containing an “⊙,” such as loop 41b, may generated magnetic fields directed in the opposite direction. Note that these magnetic field directions are based on the direction of current via the leads 33a, 33b and may be reversed if the current direction is reversed. In particular, to create the aforementioned condition in which adjacent sections have opposite electromagnetic field directions, the wiring pattern may be created such that adjacent sections of adjacent wire loops have loop current flowing in opposite directions. While FIG. 4 shows the loops having a square shape, the shape of the loops is not thus limited, and for example, circular shapes or non-square polygonal shapes may be used.

In utilizing the system, the distance of the array to the patient may be adjusted to minimize safety concerns, such as implanted pacemakers, spinal cord stimulators, and surgical implants (particularly noting the distance-cubed inverse relationship between distance and power). Power may be adjusted by varying the pulse width, pulse frequency, peak power, distance, and number of wires in each loop, as well as the size and number of wire loops. A temperature sensor monitor may also be incorporated with software designed to turn the device off in instances where there may be concerns to prevent excessive heating. For example, a maximum temperature value may be set, and a comparison device, such as but not limited to a comparator, or software (run on a processor) written to perform such a comparison on digitized values of the measured temperature and the maximum temperature (in such a case, the measured temperature may be digitized using an analog-to-digital converter), may be used to compare the measured temperature with the maximum temperature. In a variation, a minimum patient temperature may also be predetermined, and a second comparison device, or comparison software, may be used to compare the measured temperature of the patient with the minimum temperature to turn on the device. Positioning of the array may be individualized, such as use beneath the posterior thorax with patients in the supine position who do not have pacemakers but do have total hip replacements or other metal which may be a concern with diathermy.

Another safety concern may relate to patients having metal implants (e.g., but not limited to, bone pins or plates and metal sutures). Short-wave diathermy may cause metal implants to heat up to the point of burning nearby tissue. To prevent this, the system may be equipped with a metal detection device to detect metal implants. Should metal implants be detected in the patient, adjustments may be made (e.g., use of radiation shielding materials in the region(s) of the metal implant(s) or locating the array such that it does not radiate in the region(s) of the metal implant(s)) or the system may not be used (e.g., if the patient has metal implants that are such that they prevent effective warming of the patient using short-wave diathermy).

The tissues being treated by the diathermy array may become warm and dissipate the resulting heat energy to adjacent tissues. In addition, the arterial blood that supplies these tissues may leave through the veins at an elevated temperature and distribute this warmth systemically by the cardiovascular system.

The array may be manufactured to be embedded in a gel cushion that may be placed under the surgical patient and may be made of materials that can be sterilized and reused. The connections between array 32 and signal generator (diathermy driver) 31 may be distant from the array 32, which may aid in minimizing risk of electrical injury. The placement underneath the patient may provide full access to surgical sites without impairment to staff.

Various aspects of the disclosure have been presented above. However, the invention is not intended to be limited to the specific aspects presented above, which have been presented for purposes of illustration. Rather, the invention extends to functional equivalents as would be within the scope of the appended claims. Those skilled in the art, having the benefit of the teachings of this specification, may make numerous modifications without departing from the scope and spirit of the invention in its various aspects.

What is claimed is:

1. A diathermy heating system including:
a diathermy driver; and
a diathermy array electrically coupled to the short-wave diathermy driver,
wherein the diathermy array comprises an array of wire loops.

2. The diathermy heating system of claim 1, wherein the diathermy driver comprises a short-wave diathermy driver.

3. The diathermy heating system of claim 2, wherein the short-wave diathermy driver uses a frequency of approximately 27 MHz, a pulse width of 20-400 μsec, a pulse frequency of 10-800 Hz, a peak power of a maximum of 200 W, and a maximum average power of 64 W.

4. The diathermy heating system of claim 1, wherein the diathermy driver operates is capable of supporting both a pulsed mode and a continuous mode.

5. The diathermy heating system of claim 1, wherein the wire loops are circular in shape.

6. The diathermy heating system of claim 1, wherein the wire loops are polygonal in shape.

7. The diathermy heating system of claim 6, wherein the wire loops are square in shape.

8. The diathermy heating system of claim 1, wherein the wire loops are oriented such that adjacent sections of adjacent wire loops have loop currents flowing in opposite directions.

9. The diathermy heating system of claim 1, wherein the diathermy array is embedded in a gel cushion.

10. The diathermy heating system of claim 1, further comprising a temperature sensor monitor configured to monitor a temperature of the patient and coupled to control the diathermy driver.

11. A method of patient warming, including:
applying electromagnetic radiation to the patient by using a diathermy heating system including a diathermy array.

12. The method of claim 11, further including adjusting a distance between the diathermy array and the patient.

13. The method of claim 11, further including positioning the diathermy array relative to the patient's body based on devices or substances within the patient's body.

14. The method of claim 11, wherein applying electromagnetic radiation comprises setting a diathermy driver to apply short-wave radiation at a frequency of approximately 27 MHz frequency in a pulsed mode or in a continuous mode, with a maximum peak power of 200 W and a maximum average power of 64 W.

15. The method of claim 14, wherein, in the pulsed mode, setting the diathermy driver to apply short-wave radiation comprises setting the diathermy driver to apply pulsed short-wave radiation at a pulse rate of 20-400 μsec and a pulse frequency of 10-800 Hz.

16. The method of claim 11, further including arranging the diathermy array beneath the patient.

17. The method of claim 11, wherein the electromagnetic radiation comprises electrical and magnetic fields that alternate in direction in adjacent regions of the diathermy array.

18. The method of claim 11, further including providing electromagnetic shielding to regions of the patient's body containing one or more metal implants.
